(19) United States
(12) Patent Application Publication
(10) Pub. No.: US 2013/0006003 A1
(43) Pub. Date: Jan. 3, 2013

(54) NEW SYNTHONES FOR PREPARATION OF 19-NOR VITAMIN D DERIVATIVES

(75) Inventors: Michal Chodynski, Warszawa (PL); Malgorzata Krupa, Warszawa (PL); Krzysztof Krajewski, Warszawa (PL); Marek Kubiszewski, Ilow (PL); Andrzej Kutner, Warszawa (PL); Anita Pietraszek, Warszawa (PL); Kinga Trzcinska, Garwolin (PL)

(73) Assignee: INSTYTUT FARMACEUTYCZNY, Warszawa (PL)

(21) Appl. No.: 13/389,291

(22) PCT Filed: Aug. 7, 2010
(86) PCT No.: PCT/PL2010/000069

§ 371 Date: Jul. 11, 2012

(30) Foreign Application Priority Data
Aug. 7, 2009 (PL) P-388752

(51) Int. Cl. C07C 401/00; C07F 7/18; C07C 317/14

(52) U.S. Cl. 552/653; 568/31

(57) ABSTRACT

The present invention discloses the synthone of Formula (I), wherein R₁ and R₂ are the same or different and represent independently hydrogen atom or hydroxyl protecting group, and its use for preparation of 19-nor vitamin D derivatives of general Formula (IV), wherein [symbol] represents single or double bond, p represents an integer 0 to 3, R₁ and R₂ represent independently hydrogen atom or hydroxyl protecting group, R₃ represents hydrogen atom, CH₃ or hydroxyl group, R₄, R₅ and R₆ represent independently hydrogen atom, C₁-C₃-alkyl or hydroxyl group or two of R₄, R₅ and R₆ substituents altogether form cyclopropyl group, in particular for preparation of paricalcitol.

### FIELD OF INVENTION

The present invention relates to the new synthones for preparation of 19-nor vitamin D derivatives with pharmaceutical activity, in particular for preparation of paricalcitol.

### BACKGROUND OF THE INVENTION

1α-Hydroxylated vitamin D derivatives, in particular 1α,25-dihydroxy vitamin D₃ and 1α,25-dihydroxy vitamin D₂, act significant role as the regulators in homeostasis and cell differentiation in humans and animals. A number of structure analogues of these metabolites, which differ in side chain structure, hydroxyl groups position and stereochemistry, were successfully applied in psoriasis, osteodystrophy, rachitis and osteoporosis treatment as well as in many other malicious diseases therapy.

European patent specification EP 387077 B1 discloses a new class of biologically active 1α-hydroxy vitamin D derivatives in which the ring A exocyclic C-19 methylene group at C-10 position, typical of natural vitamin D structure has been replaced by two hydrogen atoms. This class of 19-nor vitamin D compounds comprises, inter alia, (1R,3R,7E,22E)-19-nor-9,10-secoergosta-5,7,22-trieno-1,3,25-triol represented by the chemical structure depicted below:

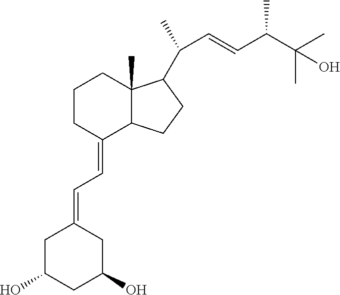 [SMILES: C[C@H](/C=C/[C@H](C)C(C)(C)O)C1CCC2/C(=C/C=C3C[C@@H](O)C[C@H](O)C3)CCC[C@@]21C]

(1R,3R,7E,22E)-19-Nor-9,10-secoergosta-5,7,22-trieno-1,3,25-triol, known under the International Non-proprietary Name paricalcitol, inhibits biosynthesis and secretion of parathormone and is approved for the treatment of secondary hyperparathyroidism due to chronic renal failure. It may be also useful as therapeutic agent for treatment of osteodystrophy, tumor, coronary arterial diseases and psoriasis.

There are two main synthetic strategies towards 19-nor vitamin D preparation. The first one is based on using naturally occurring vitamin D derivatives, from which after multistep synthetic transformations the final compound of desired structure is obtained. The alternate strategy is the total synthesis, in which functionalized synthones are combined together to yield vitamin D derivative of desired structure.

Synthesis of 19-nor vitamin D derivatives disclosed in EP 387077 B1 comprises the use of highly advanced synthone, which is naturally occurring 25-hydroxy vitamin D₂. This synthone is subject to multi-step synthetic transformations, among which cyclisation, 1α-hydroxylation and 10-dehydroxylation are the most crucial.

The total synthesis strategy is described in the European patent specification EP 582481 B1. Synthesis of 1α-hydroxy-19-nor vitamin D described in this patent is based on the construction of 5,8-diol-6-yn as the key intermediate obtained in the condensation of cyclic dihydroxyketone (precursor of ring A) and acetylenide derivative (synthone CD). The latter is expected to have the side chain of the same structure as the one of the final product. Partial reduction of acetylenic bond and removal of hydroxyl groups at C-5 and C-8 positions under reductive conditions, yields 5,7-diene as a mixture of 7,8-cis and 7,8-trans isomers. The desired (7E) isomer is either directly separated from the reaction mixture, followed by chromatography purification, or it is obtained due to isomerization of (7Z) isomer.

Both intermediates and the final product obtained according to the described methods, require separation and purification by chromatography, therefore none of the prior art synthetic routes is suitable in a big scale production of 19-nor vitamin D derivatives.

European patent specification EP 516411 B1 discloses important synthones useful in 19-nor vitamin D convergent synthesis, bearing hydroxymethyl, carbaldehyde or carboxyl group at C-22 position. They are obtained in the Wittig-type reaction, by coupling appropriate phosphine oxide, which is a ring A synthone, with bicyclic ketone—a ring CD precursor of vitamin D carbon skeleton. These synthones can be used in synthetic transformations yielding 19-nor vitamin D derivatives, either due to incorporation of the side chain of the final product structure or its further synthetic modifications. As the example, Grignard reaction is described of 3-hydroxy-3-methylbutyl magnesium bromide with protected hydroxyl group and C-22-tosyl derivative of 9,10- seco-19,22,23-trinorchola-5,7-dien-1,3-diol, which results in obtaining protected derivative of 1α,25-dihydroxy-19-nor-vitamin D₃.

Now, it has been found that the new synthone, (7E)-(1R,3R)-24-phenylsulfonyl-9,10-seco-19,22,23-trinorchola-5,7-dien-1,3-diol, may be successfully employed in the synthesis of biologically active 19-nor vitamin D derivatives, including paricalcitol. The new synthone may be obtained following standard synthetic transformations using easily available substrate, which is naturally occurring vitamin D₂. Unexpectedly it was found that the use of the new synthone enables preparation of 19-nor vitamin D derivatives of high purity, avoiding troublesome preparative chromatography purification of succeeding intermediates. The new synthone and its precursor, (7aR)-7a-methyl-1-((S)-1-(phenylsulfonyl)propan-2-yl)-hexahydro-1H-inden-4(2H)-on, easily crystallize in organic solvents, therefore most of the impurities generated in the prior steps of synthesis can be eliminated during the crystallization process.

The new synthone, (7 E)-(1R,3R)-24-Phenylsulfonyl-9,10-seco-19,22,23-trinorchola-5,7-dien-1,3-diol, may be useful in the process for preparation of 19-nor vitamin D derivatives relaying on the incorporation of the side chain of 19-nor vitamin D structure at position C-22 of vitamin D carbon skeleton. One of the known synthetic methods of introducing unsaturated carbon chains into the molecule is olefination under Marc Julia protocol (Tetrahedron Lett. 1973, 4833). In that method, phenylsulfonyl anions, generated upon treatment with n-butyl lithium, are reacted with aldehydes, the resulting intermediates are subject to functionalisation and reductive elimination to furnish alkenes. Another synthetic approach, applied for synthesis of vitamin D derivatives with alkyl saturated side chain, comprises nucleophilic addition of alkylating agents, such as sulfonates, alkyl halogens (H. O. House “Modern Synthetic Reactions”, W. A. Benjamin, Inc., Menlo Park, Calif., USA, 1972; Chapter 9) or chiral epoxides having expected vitamin D configuration, and subsequent reductive desulfonation of the intermediate (WO 99/36400).

### DISCLOSURE OF THE INVENTION

First aspect of the invention is the new synthone for the preparation of biologically active 19-nor vitamin D derivatives, represented by Formula (I),

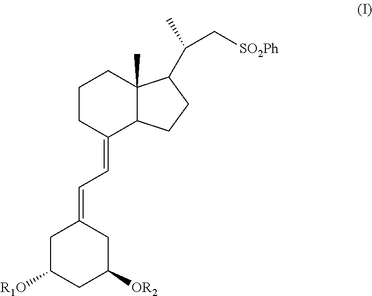

wherein R₁ and R₂ are the same or different and represent independently hydrogen atom or hydroxyl protecting group.

In the second aspect, the invention relates to the process for preparation of the synthone of Formula (I) under Horner-Wittig conditions using phosphine oxide of Formula (II), which is precursor of ring A

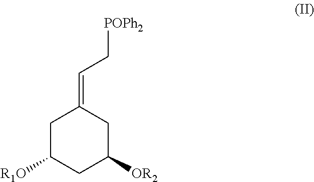

and a bicyclic ketone of Formula (III),

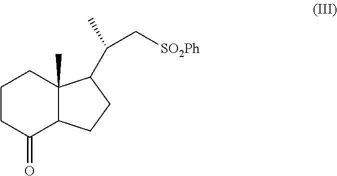

which is precursor of ring CD of the final vitamin.

The other aspect of the invention is the new compound, (7aR)-7a-methyl-1-((5)-1-(phenylsulfonyl)propan-2-yl)hexahydro-1H-inden-4(2H)-on (Formula III),

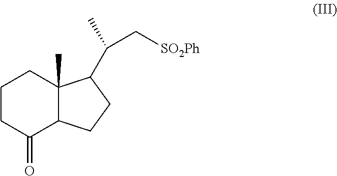

which is precursor of ring CD of the final vitamin, used for preparation of synthone of Formula (I).

In another aspect, the present invention provides the use of synthone of Formula (I), wherein R₁ and R₂ are the same or different and represent independently hydrogen atom or hydroxyl protecting group, for the preparation of biologically active 19-nor vitamin D derivatives of general Formula (IV),

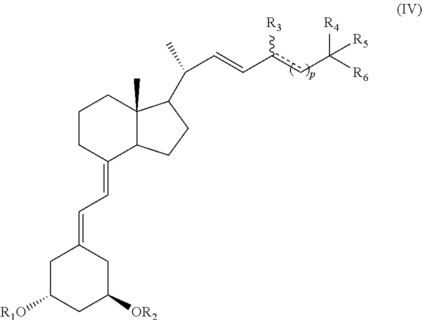

wherein [symbol] represents single or double bond, p represents an integer 0 to 3, R₁ and R₂ represent independently hydrogen atom or hydroxyl protecting group, R₃ represents hydrogen atom, CH₃ or hydroxyl group, R₄, R₅ and R₆ represent independently hydrogen atom, C₁-C₃-alkyl or hydroxyl group, or two of R₄, R₅ and R₆ substituents altogether form cyclopropyl group.

In the preferred embodiment, the present invention relates to the process for preparation of 19-nor vitamin D derivatives of general Formula (IV), wherein p represents 0, [symbol] represents single bond, R₁ and R₂ represent hydrogen atoms, R₃, R₄ and R₅ represent CH₃ group, R₆ represents hydroxyl group and C-24 has R or S configuration, having Formula (IVa),

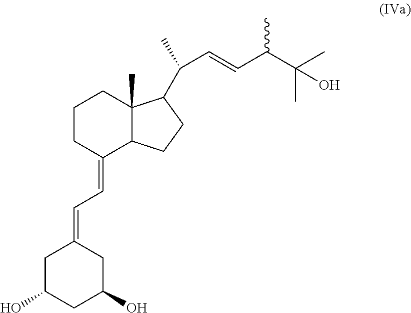

and most preferably to the process for preparation of compound of Formula (IVa), wherein C-24 has S configuration, ie. paricalcitol.

### DETAILED DESCRIPTION OF THE INVENTION

The new synthone useful in the process for preparation of biologically active 19-nor vitamin D derivatives is the compound of Formula (I), wherein R₁ and R₂ are the same or different and represent independently hydrogen atom or hydroxyl protecting group.

The protecting groups are those traditionally used to protect hydroxyl function in vitamin D chemistry, which are stable under acidic or basic conditions. They embrace such groups as, for example, alkyl- and arylsilyl; alkyl- and arylcarbonyl (ester); acyl; alkylaminocarbonyl (carbamate); alkyl; alkoxyl group and other.

The preferred protecting groups are silyl groups, such as trialkylsilyl, dialkylarylsilyl, alkyldiarylsilyl, triarylsilyl, for example trimethylsilyl, triethylsilyl, t-butyldimethylsilyl, t-butyldiphenylsilyl and triphenylsilyl group. The other preferred groups are acyl groups including alkanoyl and carboxyalkanoyl groups, having 1-6 carbon atoms, preferably acetyl group. The typical alkoxyalkyl groups are methoxymethyl, ethoxyethyl, tetrahydrofuranyl and tetrahydropyranyl groups.

Preferably, synthone of Formula (I) according to the invention represents (7E)-(1R,3R)-24-phenylsulfonyl-9,10-seco-19,22,23-trinorchola-5,7-dien-1,3-diol or its derivative protected with t-butyldimethylsilyl group, ie. (7E)-(1R,3R)-24-phenylsulfonyl-1,3-bis(t-butyldimethylsilyloxy)-9,10-seco-19,22,23-trinorchola-5,7-dien.

Protection and deprotection of hydroxyl groups are procedures well known to those skilled in the art and described, inter alia, in T. W. Greene, P. G. M. Wuts “Protective Groups in Organic Synthesis”, ed. 3, John Wiley and Sons, Inc., New York, N.Y., 1999; P. J. Kocienski “Protecting Groups”, Georg Thieme Verlag, Stuttgart, 2004; J. March, Advanced Organic Chemistry”, John Wiley and Sons, New York, N.Y., 1992. Synthone of Formula (I) is obtained under Horner-Wittig conditions in the reaction of phosphine oxide of Formula (II) and (7aR)-7a-methyl-1-((S)-1-(phenylsulfonyl)propan-2-yl)hexahydro-1H-inden-4(2H)-on (III).

The example of the reaction with the use of the compound of Formula (II), (3R,5R)-[bis(tert-butyldimethyl-silyloxy)]cyclohexylidene ethyldiphenylphosphine oxide, is depicted on Scheme 1.

Preparation of the starting compound, (3R,5R)-[bis(tert-butyldimethylsilyloxy)]cyclohexylidene ethyldiphenylphosphine oxide, may be accomplished according to the procedure disclosed in EP 516411 B1, with the use of commercially available (1R,3R,4R,5R)-(−)-quinic acid having the same configuration at C1-OH and C3-OH as the final 19-nor vitamin D.

(7aR)-7a-Methyl-1-((S)-1-(phenylsulfonyl)propan-2-yl)hexahydro-1H-inden-4(2H)-on may be obtained following standard procedure depicted on Scheme 2. Synthetic methods of preparation of this class of compounds can be found in the literature, for example J. Org. Chem. 51, 3098 (1986), J. Org. Chem. 1986, 1264 (1986) and other. Thus, vitamin D₂ upon ozonolyzis is converted into diol, which following subsequent transformations yields hydroxyiodide, than ketoiodide and finally (7aR)-7a-Methyl-1-((S)-1-(phenylsulfonyl)propan-2-yl)hexahydro-1H-inden-4(2H)-on.

(7aR)-7a-Methyl-1-((S)-1-(phenylsulfonyl)propan-2-yl)hexahydro-1H-inden-4(2H)-on may further be subject to crystallization in organic solvent or the mixture of organic solvent and water to remove side reactions products.

It has been found that (7aR)-7a-methyl-1-((S)-1-(phenylsulfonyl)propan-2-yl)hexahydro-1H-inden-4(2H)-on crystallizes in different solvents in the same crystalline form.

Crystalline form of (7aR)-7a-methyl-1-((S)-1-(phenylsulfonyl)propan-2-yl)hexahydro-1H-inden-4(2H)-on has characteristic peaks in X-ray powder diffraction (XRPD) pattern recorded with CuKλ, λ=1,54056 Å, as presented by the following reflection angles 2θ [°], interplanar spacings d [Å] and relative intensities in attitude to the most intensive diffraction peak, I/I₀ [%] as set forth in Table 1:

TABLE 1

| d [Å] | 2θ [°] | I/I₀ [%] |
|---|---|---|
| 10.208 | 8.66 | 1.1 |
| 9.734 | 9.08 | 14.5 |
| 7.729 | 11.44 | 3.4 |
| 7.515 | 11.77 | 28.6 |
| 6.128 | 14.44 | 9.3 |
| 5.843 | 15.15 | 25.1 |
| 5.525 | 16.03 | 100 |
| 5.437 | 16.29 | 41.4 |
| 5.150 | 17.21 | 5.4 |
| 4.681 | 18.94 | 22.8 |
| 4.522 | 19.62 | 25.6 |
| 3.856 | 23.05 | 2.7 |
| 3.777 | 23.54 | 24 |
| 3.460 | 25.73 | 29 |
| 3.431 | 25.95 | 2.8 |
| 3.333 | 26.72 | 5.5 |
| 3.296 | 27.03 | 15.1 |
| 3.176 | 28.07 | 5.2 |
| 3.025 | 29.51 | 0.9 |
| 2.294 | 39.24 | 1.1 |

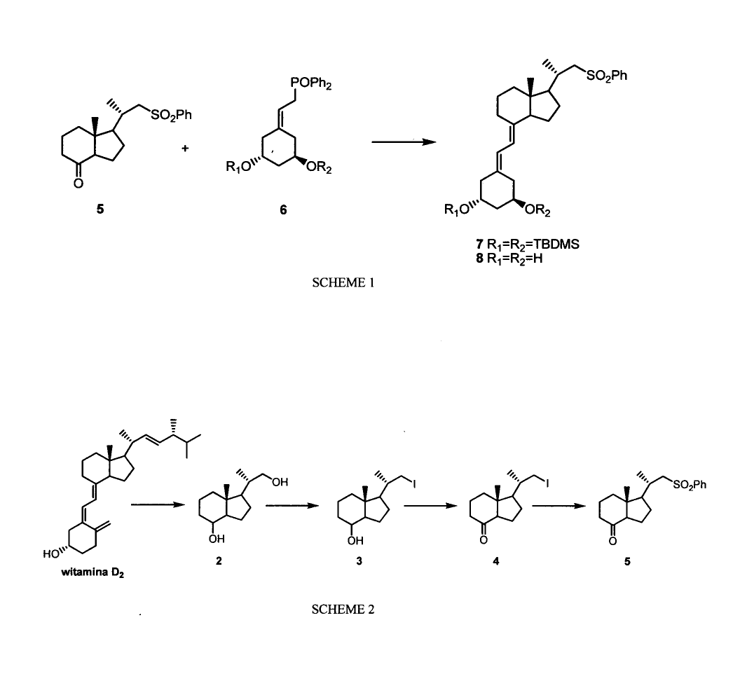

Crystalline form of (7aR)-7a-methyl-1-((S)-1-(phenylsulfonyl)propan-2-yl)hexahydro-1H-inden-4(2H)-on is further characterized by X-ray diffraction pattern (XRPD) substantially as depicted in FIG. 1.

The reaction of phosphine oxide and (7aR)-7a-methyl-1-((S)-1-(phenylsulfonyl)propan-2-yl)hexahydro-1H-inden-4(2H)-on is preformed in aprotic solvent in the presence of a strong base such as alkali metal hydroxide, alkyl or aryl lithium or alkylamide lithium, preferably n-butyl lithium.

After completion of the reaction, protecting groups, if necessary, are removed under standard conditions, and obtained (7E)-(1R,3R)-24-phenylsulfonyl-9,10-seco-19,22,23-trinorchola-5,7-dien-1,3-diol is separated using chromatography technique.

(7E)-(1R,3R)-24-Phenylsulfonyl-9,10-seco-19,22,23-trinorchola-5,7-dien-1,3-diol crystallizes easily in organic solvents. The crystalline solid has characteristic peaks in X-ray powder diffraction (XRPD) pattern recorded with CuKα, λ=1,54056 Å, as presented by the following reflection angles 2θ [°], interplanar spacings d [Å] and relative intensities in attitude to the most intensive diffraction peak, I/I₀ [%] as set forth in Table 2:

TABLE 2

| d [Å] | 2θ [°] | I/I₀ [%] |
|---|---|---|
| 11.144 | 7.93 | 2.5 |
| 8.486 | 10.42 | 22.2 |
| 7.513 | 11.77 | 4.7 |
| 5.787 | 15.30 | 19.8 |
| 5.633 | 15.72 | 21.2 |
| 5.163 | 17.16 | 100 |
| 4.912 | 18.04 | 30.2 |
| 4.500 | 19.71 | 30.3 |
| 4.270 | 20.79 | 10.3 |
| 4.048 | 21.94 | 19.3 |
| 3.929 | 22.61 | 12.3 |
| 3.709 | 23.97 | 15.3 |
| 3.568 | 24.93 | 11.5 |
| 3.442 | 25.86 | 8.4 |
| 3.231 | 27.59 | 10.1 |
| 3.037 | 29.39 | 5.6 |
| 2.749 | 32.55 | 3.1 |
| 2.539 | 35.32 | 3.8 |
| 2.447 | 36.70 | 2.3 |

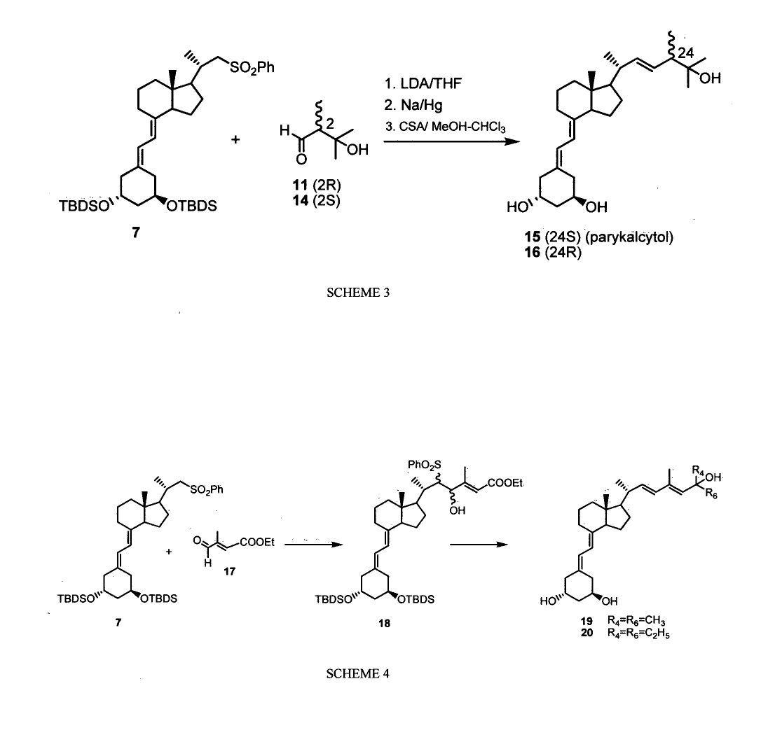

Crystalline form of (7E)-(1R,3R)-24-phenylosulfonyl-9,10-seco-19,22,23-trinorchola-5,7-dien-1,3-diol is further characterized by X-ray diffraction pattern (XRPD) substantially as depicted in FIG. 2.

Single crystallization of (7E)-(1R,3R)-24-phenylsulfonyl-9,10-seco-19,22,23-trinorchola-5,7-dien-1,3-diol yields the compound of high chemical purity (>99%, HPLC) that can be used directly, following optional hydroxyl groups protection, in the synthesis of 19-nor vitamin D derivatives of general Formula (IV).

The process for preparation of 19-nor vitamin D derivatives of general Formula (IV),

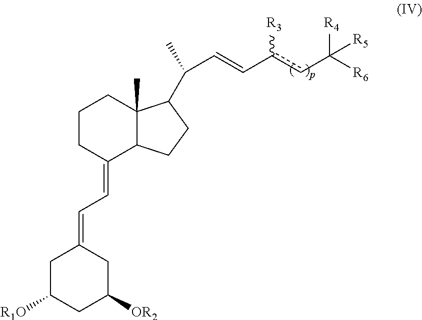

wherein [symbol] represents single or double bond, p represents an integer 0 to 3, R₁ and R₂ represent independently hydrogen atom or hydroxyl protecting group, R₃ represents hydrogen atom, CH₃ or hydroxyl group, R₄, R₅ and R₆ represent independently hydrogen atom, C₁-C₃-alkyl or hydroxyl group or two of R₄, R₅ and R₆ substituents altogether form cyclopropyl group, comprises:
(i) reacting synthone of Formula (I),

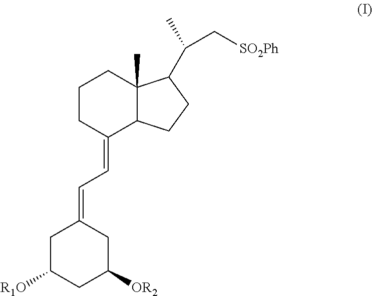

wherein R₁ and R₂ are the same or different and represent hydroxyl protecting groups,
with aldehyde of Formula (Va) or (Vb),

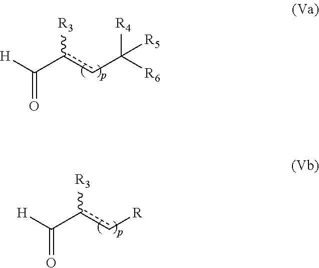

wherein p represents an integer 0 to 3, [symbol] represents single or double bond, R₃ represents hydrogen atom, CH₃ or hydroxyl group, R₄ and R₅ represent hydrogen atom or C₁-C₃-alkyl group, R₆ represents hydroxyl group and R represents carboxyl group or two of R₄, R₅ and R₆ substituents altogether form cyclopropyl group, in the presence of strong organic base in aprotic solvent, to obtain the mixture of alfa-hydroxysulfones,
(ii) reductive elimination of the adjacent phenylsulfonyl and hydroxyl groups from the mixture of alfa-hydroxysulfones obtained in step (ii) with sodium amalgam, to obtain the product of olefination of Formula (IVa) or (IVb), respectively,

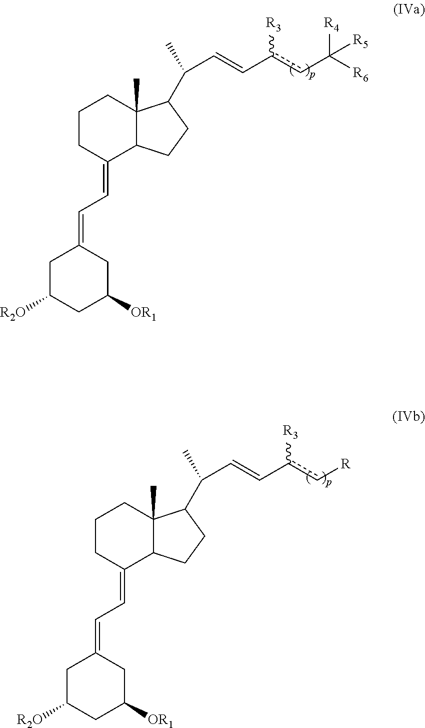

wherein R₁-R₆, R and p have the meaning as defined above,
(iii) optionally, standard synthetic modification of the side chain of the compound of Formula (IVb), to obtain the compound of Formula (IVa), wherein R₁-R₆ and p have the meaning as defined above,
(iv) optionally, removing the hydroxyl protecting groups and purification of the product.

Reaction of synthone of Formula (I) with aldehyde of Formula (Va) or (Vb) is accomplished with the use of a strong base generating an anion of phenylsulfonyl derivative (I), preferably organometallic compound, such as lithium, sodium or potassium organic compound, such as n-butyl lithium, in the presence of a base, for example N,N-diisopropylamine, in aprotic solvents, such as phosohoric acid alkyl amides or urea alkyl derivatives, preferably hexamethylphosphorous triamide (HMPT) or tetrahydrofurane (THF).

In dehydroxy-desulfonation reaction, commercially available 5 or 10% sodium amalgam is used.

Deprotection of hydroxyl groups is carried out under basic conditions. Typically used silyl groups are removed, for example, upon treatment with tetrabutylammonium fluoride in organic solvent, such as THF or acetone, optionally in presence of water.

Use of synthone of Formula (I) for preparation of 19-nor vitamin D derivatives of general Formula (IV), wherein p represents 0, [symbol] represents single bond, R₁ and R₂ represent hydrogen atoms, R₃, R₄ and R₅ represent CH₃ groups, R₆ represents hydroxyl group, R or S configuration at C-24 of end vitamin D is the same as configuration of (2R)- or (2S)-3-hydroxy-2,3-dimethylobutanal (Formula (Va), p=0, R₄=R₆=H, R₅=OH), is illustrated by the exemplary synthesis of paricalcitol and its (24R) isomer of Formula (IVa) depicted on Scheme 3.

Another example of preparation of 24a-homo-19-nor vitamin D of Formula (IV), wherein p represents 1, [symbol] represents double bond, R₁ and R₂ represent hydrogen atoms, R₃ represents CH₃ group, R₅ represents hydroxyl group, R₄ and R₆ represent C₁-C₃-alky groups is depicted on Scheme 4. These derivatives can be obtained in the reaction of synthone of Formula (I) and aldehyde-ester of Formula (Vb), wherein R represents carboxyl group, for example ethyl 3-methyl-4-oxobut-2-enate. During dehydroxy-desulfonation reaction, phenylsulfonyl moiety along with neighboring hydroxyl group are eliminated and obtained ester is subject to reaction with appropriate C₁-C₃-alkyl magnesium bromide under Grignard protocol.

The present invention provides efficacious process for preparation of 19-nor vitamin D derivatives with optional side chain in direct synthesis, said process based on the use of synthone of Formula (I). The process according to the invention enables vitamin D derivatives preparation without both diastereoisomeric mixture separation as well as laborious purification of the intermediates.

The present invention is further illustrated by the following examples.

### EXAMPLES

### Analytical Methods

The product was dried in the vacuum drier Salvis Lab VC-20 (Donserv).

¹H-NMR and ¹³C-NMR spectra were recorded on GEMINI-200 Varian spectrometer.

Ultraviolet spectra were obtained using Shimadzu UV-160A spectrophotometer.

X-ray powder diffraction patterns (XRPD) were recorded on the MiniFlex Rigaku powder diffractometer with copper radiation CuKα, λ=1,54056 Å, with the following measurement parameters:
2Θ scanning range: 3°-40°
Scanning rate Δω: 0.5°/min.
Step size Δ2Θ: 0.03°
Measurement temperature: room temperature
Detector: scintillation counter

Obtained diffraction patterns were work out and analyzed using DHN_PDS program.

### Example 1

### Preparation of (7aR)-1-((S)-1-hydroxypropane-2-yl)-7a-methyloctahydro-1H-inden-4-ol (2)

Vitamin D₂ (400 g) in the solution of dichloromethane (4 L) and methanol (12 L) was placed in the reaction vessel. The mixture was stirred until the whole amount of solid vitamin was dissolved, than the solution was cooled down to about −70° C. at acetone/CO₂ cooling bath. Ozone was purged for 8 h (at about 0.3 m³/h rate) until the solution turned blue. Obtained ozonides were reduced with NaBH₄ (308 g), at temperature range from −50° C. to 20° C. within 20 h. To the reaction mixture saturated brine solution (4 L), 2 M aqueous HCl solution (3.4 L) and dichloromethane (4 L) were added, respectively. Organic phase was separated and water layer was extracted with one portion of dichloromethane (4×2 L). Combined organic layers were concentrated to dryness under vacuum (temp. of heating bath 35±5° C.). Obtained crude product was dissolved in toluene (2 L), the solution was concentrated under reduced pressure to half of its volume, and the mixture was placed in the chromatography column. Obtained eluate was concentrated to dryness. To the solid product hexane (0.6 L) was added. The mixture was stirred at RT for 2 h. The solution was filtered. Obtained solid was dried in a vacuum drier at temp. 30° C. for 4 h. Diol 2 was obtained in 148 g (68%) yield.

### Example 2

### Preparation of (7aR)-1-((S)-1-iodopropane-2-yl)-7a-methyloctahydro-1H-inden-4-ol (3)

In a round-bottom flask (1000 mL) triphenylophosphine (24.7 g), dichloromethane (300 mL), triethylamine (26.0 mL) and iodide (24.0 g) were placed. The mixture was stirred for 30 min., than diol 2 (20.0 g) solution in dichloromethane (100 mL) was added. Stirring was continued for 20 h. To the reaction mixture silica gel (60 g) was added and the solvent was removed under reduced pressure. The residue was purified by silica gel chromatography (hexane—ethyl acetate gradient from 5% to 20%). Iodide 3 was obtained in 24.7 g (82%) yield.

### Example 3

### Preparation of (7aR)-1-(S)-1-iodopropane-2-yl)-7a-methylhexahydro-1H-inden-4(2H)-on (4)

In a round-bottom flask (1000 mL) pyridinium chlorochromate (PCC) (15.7 g), celit (15 g) and dichloromethane (300 mL) were placed. The mixture was stirred for 30 min. Iodide 3 (6.0 g) in dichloromethane (30 mL) solution was added and stirring was continued at RT for 1 h. The reaction mixture was filtered through silica gel layer. Solvent was removed under reduced pressure. The crude product was purified by silica gel chromatography (hexane—ethyl acetate 50%). Iodoketone 4 was obtained in 4.7 g (78%) yield.

### Example 4

### Preparation of (7aR)-7a-methyl-1-((S)-1-(phenylsulfonyl)propane-2-yl)hexahydro-1H-inden-4(2H)-on (5)

In a round-bottom flask (1000 mL) iodoketone 4 (4.7 g), DMF (30 mL) and sodium benzenesulfinate (11.0 g) were placed. The mixture was stirred at temp. 80° C. for 90 min. The solution was cooled down, water (20 mL) was added and the product was extracted with ethyl acetate (5×25 mL). Organic layers were dried over anh. MgSO₄, the solvents were removed under reduced pressure. The residue was purified by silica gel chromatography (hexane—ethyl acetate gradient from 10% to 70%). Sulfone 5 (4.0 g) was obtained. The product was crystallized in boiling ethyl acetate (25 mL). Crystalline sulfone 5 was obtained in 3.4 g (70%) yield.

¹H-NMR (200 MHz, CDCl₃); S 0.61 (s, 3H, CH₃), 1.25 (d, J=6.7 Hz, 3H, CH₃), 3.02 (m, 2H, CH₂), 7.52-7.93 (m, 5H, —Ar); ¹³C-NMR (200 MHz, CDCl₃) 12.24, 18.87, 20.04, 23.2, 27.13, 31.96, 38.54, 40.69, 49.62, 55.59, 61.59, 127.72, 129.26, 133.59, 140.08, 211.09.

### Example 5

Crystallization of (7aR)-7a-methyl-1-((S)-1-(phenylsulfonyl)propane-2-yl)hexahydro-1H-inden-4(2H)-on (5).

### a) Crystallization in Acetone

In a round-bottom flask (25 mL) sulfone 5 (1.00 g) and acetone (7.0 mL) were placed. The mixture was heated under reflux, until the solution became clear. The solution was left at temp. 20° C. for 48 h. The crystalline product was filtered off on Buchner funnel and washed with cold acetone (5 mL) The product was dried in a vacuum drier (10 mbar, 40° C.) to dry mass. Crystalline sulfone 5 was obtained in 0.85 g yield.

XRPD (2θ), [°]: 8.60, 8.79, 9.10, 11.56. 11.77, 14.45, 15.21, 16.01, 16.24, 17.22, 18.86, 19.59, 23.08, 23.50, 25.65, 25.98, 26.64, 26.97, 28.03, 29.31, 39.15; DSC: endothermic peak 154.61° C. (onset 153.72° C.)

### b) Crystallization in a Mixture of Acetone-Water

In a round-bottom flask (25 mL) sulfone 5 (1.00 g), acetone (7.0 mL) and water (1.0 mL) were placed. The mixture was heated under reflux, until the solution became clear. The solution was left a temp. 20° C. for 48 h. The crystalline product was filtered off on Büchner funnel and washed with cold acetone (5 mL). The product was dried in a vacuum drier (10 mbar, 40° C.) to dry mass. Crystalline sulfone 5 was obtained in 0.80 g yield.

XRPD (2θ), [°]: 8.64, 9.15, 11.30. 11.62, 14.30, 15.20, 15.90, 16.29, 17.29, 18.94, 19.60, 23.10, 23.43, 25.64, 25.98, 26.68, 27.03, 28.11, 29.55, 39.25; DSC: endothermic peak 154.98° C. (onset 154.10° C.)

### c) Crystallization in a Mixture of Acetone-Hexane

In a round-bottom flask (25 mL) sulfone 5 (1.00 g), acetone (4.0 mL) and hexane (2.0 mL) were placed. The mixture was heated under reflux, until the solution became clear. The solution was left at temp. 20° C. for 48 h. The crystalline product was filtered off on Büchner funnel and washed with cold acetone (5 mL). The product was dried in a vacuum drier (10 mbar, 40° C.) to dry mass. Crystalline sulfone 5 was obtained in 0.78 g yield.

XRPD (2θ), [°]: 8.61, 9.11, 11.23, 11.75, 14.38, 15.20, 15.94, 16.23, 17.19, 18.90, 19.55, 23.02, 23.42, 25.56, 25.99, 26.59, 26.93, 28.04, 29.49, 39.86; DSC: endothermic peak 154.95° C. (onset 154.09° C.)

### d) Crystallization in a Mixture of Acetone-t-Butyl Methyl Ether)

In a round-bottom flask (25 mL) sulfone 5 (1.00 g), acetone (4.0 mL) and t-butyl methyl ether (2.0 mL) were placed. The mixture was heated under reflux, until the solution became clear. The solution was left at temp. 20° C. for 48 h. The crystalline product was filtered off on Büchner funnel and washed with cold acetone (5 mL). The product was dried in a vacuum drier (10 mbar, 40° C.) to dry mass. Crystalline sulfone 5 was obtained in 0.81 g yield.

XRPD (2θ), [°]: 8.61, 9.05, 11.40. 11.75, 14.39, 15.23, 16.02, 16.24, 17.24, 18.87, 19.53, 23.07, 23.46, 25.55, 25.85, 26.57, 26.89, 28.01, 29.48, 39.17; DSC: endothermic peak 154.41° C. (onset 153.29° C.)

### e) Crystallization in Acetonitrile

In a round-bottom flask (25 mL) sulfone 5 (1.00 g) and acetonitrile (7.0 mL) were placed. The mixture was heated under reflux, until the solution became clear. The solution was left at temp. 20° C. for 48 h. The crystalline product was filtered off on Buchner funnel and washed with cold acetone (5 mL) The product was dried in a vacuum drier (10 mbar, 40° C.) to dry mass. Crystalline sulfone 5 was obtained in 0.88 g yield.

XRPD (2θ), [°]: 8.64, 9.03, 11.38. 11.72, 14.39, 15.17, 15.95, 16.26, 17.22, 18.85, 19.54, 23.01, 23.49, 25.69, 25.99, 26.73, 26.96, 27.99, 29.43, 39.18; DSC: endothermic peak 154.47° C. (onset 152.97° C.)

f) Crystallization in 1,2-Dimethoxyethane

In a round-bottom flask (25 mL) sulfone 5 (1.00 g) and 1,2-dimethoxyethane (7.0 mL) were placed. The mixture was heated under reflux, until the solution became clear. The solution was left a temp. 20° C. for 48 h. The crystalline product was filtered off on Büchner funnel and washed with cold 1,2-dimethoxyethane (5 mL). The product was dried in a vacuum drier (10 mbar, 40° C.) to dry mass. Crystalline sulfone 5 was obtained in 0.65 g yield.

XRPD (2θ), [°]: 8.66, 9.11, 11.56, 11.80, 14.17, 14.44, 15.19, 16.03, 16.23, 17.26, 18.73, 19.64, 23.09, 23.50, 25.74, 25.96, 26.87, 27.09, 27.96, 29.33, 39.13; DSC: endothermic peak 154.83° C. (onset 154.38° C.)

### g) Crystallization in Toluene

In a round-bottom flask (25 mL) sulfone 5 (1.00 g) and toluene (7.0 mL) were placed. The mixture was heated under reflux, until the solution became clear. The solution was left a temp. 20° C. for 48 h. The crystalline product was filtered off on Büchner funnel and washed with cold toluene (5 mL). The product was dried in a vacuum drier (10 mbar, 40° C.) to dry mass. Crystalline sulfone 5 was obtained in 0.74 g yield.

XRPD (2θ), [°]: 8.62, 9.13, 11.37. 11.67, 14.42, 15.20, 15.98, 16.22, 17.21, 18.78, 19.56, 23.02, 23.49, 25.62, 25.95, 26.60, 26.86, 28.04, 29.49, 39.28; DSC: endothermic peak 154.26° C. (onset 153.49° C.)

### h) Crystallization in Methanol

In a round-bottom flask (25 mL) sulfone 5 (1.00 g) and methanol (8.0 mL) were placed. The mixture was heated under reflux, until the solution became clear. The solution was left a temp. 20° C. for 48 h. The crystalline product was filtered off on Büchner funnel and washed with cold methanol (5 mL). The product was dried in a vacuum drier (10 mbar, 40° C.) to dry mass. Crystalline sulfone 5 was obtained in 0.82 g yield.

XRPD (2θ), [°]: 8.58, 9.10, 11.36. 11.77, 14.43, 15.19, 15.89, 16.23, 17.24, 18.86, 19.56, 22.96, 23.52, 25.66, 25.90, 26.60, 26.92, 28.04, 29.34, 39.21; DSC: endothermic peak 154.68° C. (onset 154.17° C.)

### i) Crystallization in Ethyl Acetate

In a round-bottom flask (50 mL) sulfone 5 (1.00 g), ethyl acetate (15.0 mL) were placed. The mixture was heated under reflux, until the solution became clear. The solution was left a temp. 20° C. for 48 h. The crystalline product was filtered off on Büchner funnel and washed with cold ethyl acetate (5 mL). The product was dried in a vacuum drier (10 mbar, 40° C.) to dry mass. Crystalline sulfone 5 was obtained in 0.90 g yield.

XRPD (2θ), [°]: 8.66, 9.08, 11.44. 11.77, 14.44, 15.15, 16.03, 16.29, 17.21, 18.94, 19.62, 23.05, 23.54, 25.73, 25.95, 26.72, 27.03, 28.07, 29.51, 39.24; DSC: endothermic peak 154.86° C. (onset 154.24° C.)

### Example 6

### Preparation of (7E)-(1R,3R)-24-phenylsulfonyl-1,3-bis(tert-butyldimethylsilyloxy)-9,10-seco-19,22,23-trinorchola-5,7-dien (7)

In a round-bottom flask (10 mL) (3R,5R)-[bis(tert-butyldimethylsilyloxy)]-cyclohexylidenethyldiphenylphosphine oxide (6) (0.33 g) in THF (0.7 mL) was placed. The solution was cooled down to 0° C. and n-BuLi (1.6 M in hexane) (0.47 mL) was added while stirring. The resulting mixture was cooled down to −60° C. and sulfone 5 (0.11 g) in THF (0.7 mL) was added dropwise. Stirring was continued for 2 h at temp. −40° C. The reaction was quenched with brine (1 mL) addition and the product was extracted with the mixture of hexane—ethyl acetate 20% (3×10 mL). Organic layer was dried over anh. MgSO₄ (2 g). After filtration and removal of solvents the residue was purified by silica gel chromatography (hexane—ethyl acetate gradient from 1% to 5%). Sulfone 7 (0.18 g) was obtained in 80% yield. UV λₘₐₓ 243.6, 251.8, 261.6 nm; ¹H-NMR (200 MHz, CDCl₃); δ 0.05 (s, 6H, 2 xSiCH₃), 0.52 (s, 3H, CH₃), 0.86 (s, 9H, SiC(CH₃)₃), 1.23 (d, J=6.6 Hz, 3H, CH₃), 3.95-4.18 (m, 2H, CH₂), 5.79 (d, J=11 Hz, 1H, ═CH), 6.18 (d, J=11 Hz, 1H, ═CH), 7.50-7.94 (m, 5H, —Ar); ¹³C-NMR (200 MHz, CDCl₃) 11.88, 18.09, 20.26, 22.07, 23.17, 25.83, 27.47, 28.48, 29.68, 32.57, 36.76, 40.29, 43.66, 45.68, 45.98, 55.75, 56.12, 62.03, 67.91, 68.05, 116.54, 121.49, 127.86, 129.23, 133.48, 134.18, 139.76, 140.38.

### Example 7

### Preparation of (7E)-(1R,3R)-24-phenylsulfonyl-9,10-seco-19,22,23-trinorchola-5,7-dien-1,3-diol (8)

In a round-bottom flask (100 mL) sulfone 7 (1.6 g) in the mixture of methanol-chloroform 1:1 (30 mL) was placed and 10-camphorsulfonic acid (0.46 g) was added. The solution was stirred at RT for 70 min. NaHCO₃ (0.46 g) was added and stirring was continued for additional 15 min. The solution was filtered and condensed. The residue was purified by silica gel chromatography (hexane—ethyl acetate gradient from 20% to 90%). Sulfone 8 (0.98 g) was obtained in 92% yield.

UV λₘₐₓ 243.2, 251.4, 261.0 nm; ¹H-NMR (200 MHz, CDCl₃); δ 0.51 (s, 3H, CH₃), 1.20 (d, J=6.2 Hz, 3H, CH₃), 3.92-4.20 (m, 2H, CH₂), 5.81 (d, J=11.4 Hz, 1H, ═CH), 6.23 (d, J=11.4 Hz, 1H, ═CH), 7.49-7.92 (m, 5H, —Ar), ¹³C-NMR (200 MHz, CDCl₃) 11.85, 20.18, 22.08, 23.20, 27.33, 28.69, 32.54, 37.09, 40.11, 42.11, 44.56, 45.75, 55.69, 56.10, 61.92, 67.11, 67.32, 115.67, 123.48, 127.82, 129.23, 131.70, 133.53, 140.19, 141.91.

Sulfone 8 was also obtained in a crystalline form. The sample of product 8 (0.4 g) was dissolved in boiling ethyl acetate (3 mL) and the mixture left for crystallization (20° C., 24 h). The crystalline product was filtered off on Büchner funnel and washed with cold ethyl acetate (2 mL). It was dried in a vacuum drier (10 mbar, 40° C.) to dry mass. Crystalline sulfone 8 was obtained in 0.31 g yield.

DSC: endothermic peak 117.35° C. (onset 115.97° C.); XRPD (2θ), [°]: 7.93, 10.42, 11.77, 15.30, 15.72, 17.16, 18.04, 19.71, 20.79, 21.94, 22.61, 23.97, 24.93, 25.86, 27.59, 29.39, 32.55, 35.32, 36.70.

### Example 8

### Preparation of (1R,3R,7E,22E)-19-nor-9,10-secoergosta-5,7,22-trieno-1,3,25-triol (15) (Parikalcitol)

In a round-bottom flask (50 mL) diisopropylamine (1.4 mL) in THF (8 mL) was placed. The mixture was cooled down to temp. −60° C. and the solution of n-butyl lithium (1.6 M) (5.9 mL) was added dropwise. After 15 min. (7E)-(1R,3R)-24-phenylsulfonyl-1,3-bis(tert-butyldimethylsilyloxy)-9,10-seco-19,22,23-trinorchola-5,7-dien (7) (2.6 g) in THF (15 mL) was added. After following 15 min. (2R)-3-hydroxy-2,3-dimethylbutanal (11) (1.8 g) was added and the resulting mixture was stirred for 60 min. at −60° C. The reaction was quenched with brine (10 mL) addition, the product was extracted with tetrahydrofurane (3×30 mL). Organic layer was dried over anh. MgSO₄. Solvents were removed under reduced pressure, the residue was dissolved in THF (4 mL) and saturated methanolic solution of Na₂HPO₄ (20 mL) was added. While vigorously stirring, sodium amalgam (about 3 g) was added portionwise, until the substrate entirely disappeared (TLC). The reaction mixture was filtered using pleated filter paper, the filtrate was condensed on vacuum rotavapour, and the residue was diluted with water (20 mL). The product was extracted with t-butyl methyl ether (3×30 mL). Organic layer was dried over anh. MgSO₄. After removal of solvents the residue was dissolved in the mixture of methanol-chloroform 1:1 (16 mL), camphorsulfonic acid (900 mg) was added and the solution was stirred at RT for 90 min. Sodium hydrogen carbonate (900 mg) was added and stirring was continued for next 15 min. The solution was filtered using pleated filter paper, the filtrate was mixed with silica gel (3 g) and the solvents were removed under reduced pressure. The residue was purified by silica gel chromatography (hexane—ethyl acetate gradient from 10% to 80%). The crude product 15 was obtained in c.a. 0.7 g yield, after crystallization in boiling ethyl acetate (about 25 mL) crystalline paricalcitol 15 (0.5 g) was obtained.

UV λₘₐₓ 243.6, 251.8, 261.4 nm; ¹H-NMR (500 MHz, CDCl₃); S 0.56 (s, 3H, CH₃), 1.01 (d, J=7.0 Hz, 3H, CH₃), 1.04 (d, J=6.8 Hz, 3H, CH₃), 1.14 (s, 3H, CH₃), 1.17 (s, 3H, CH₃), 4.05 (m, 1H, CH—OH), 4.11 (m, 1H, CH—OH), 5.34 (m, 2H, 2 x ═CH), 5.85 (d, J=11.4 Hz, 1H, ═CH), 6.31 (d, J=11.4 Hz, 1H, ═CH); ¹³C-NMR (200 MHz, CDCl₃) 12.27, 15.84, 20.83, 22.32, 23.44, 26.24, 26.92, 28.25, 28.89, 37.15, 40.33, 40.64, 42.15, 44.65, 45.69, 48.35, 56.09, 56.34, 67.17, 67.38, 72.40, 115.35, 123.76, 129.37, 131.25, 139.33, 142.76.

### Example 9

### Preparation of (1R,3R,24R,7E,22E)-19-nor-9,10-secoergosta-5,7,22-trieno-1,3,25-triol (16)

In a round-bottom flask (25 mL) diisopropylamine (0.8 mL) in THF (6 mL) was placed. The mixture was cooled down to −60° C. and the solution of n-butyl lithium (1.6 M) (3.1 mL) was added dropwise. After 15 min. (7E)-(1R,3R)-24-phenylsulfonyl-1,3-bis(tert-butyldimethylsilyloxy)-9,10-seco-19,22,23-trinorchola-5,7-dien (7) (1.4 g) in THF (15 mL) was added. After 15 min. (2S)-3-hydroxy-2,3-dimethylbutanal (14) (1.0 g) was added the resulting mixture was stirred for 60 min. at −60° C. The reaction was quenched with brine (10 mL) addition and the product was extracted with tetrahydrofurane (3×20 mL). Organic layer was dried over anh. MgSO₄. When the solvents were removed the residue was dissolved in THF (3 mL) and diluted with saturated methanolic solution of Na₂HPO₄ (14 mL). While vigorous stirring, sodium amalgam (about 2 g) was added portionwise, until disappearance of the substrate (TLC). The mixture was filtered using pleated filter paper, and the filtrate was diluted with water (10 mL). The product was extracted with t-butyl methyl ether (3×20 mL). Organic layer was dried over anh. MgSO₄. Solvents were removed under reduced pressure, the residue was dissolved in a mixture of methanol-chloroform 1:1 (10 mL), camphorsulfonic acid (500 mg) was added and the resulting mixture was stirred for 90 min. at RT. After addition of sodium hydrogen carbonate (500 mg) stirring was continued for additional 15 min. The mixture was filtered using pleated filter paper, the filtrate was mixed with silica gel (2 g), than the solvents were removed under vacuum. The residue was purified by silica gel chromatography (hexane—ethyl acetate gradient from 10% to 80%). The crude product 16 (about 0.4 g) was crystallized in boiling ethyl acetate (about 25 mL), crystalline 24R-isomer of paricalcitol (16) was obtained in 0.25 g yield.

UV λₘₐₓ 243.6, 251.8, 261.4 nm; ¹H-NMR (500 MHz, CDCl₃); δ 0.56 (s, 3H, CH₃), 1.00 (d, J=7.0 Hz, 3H, CH₃), 1.04 (d, J=6.4 Hz, 3H, CH₃), 1.13 (s, 3H, CH₃), 1.18 (s, 3H, CH₃), 4.04 (m, 1H, CH—OH), 4.11 (m, 1H, CH—OH), 5.27 (m, 1H, ═CH), 5.35 (m, 1H, ═CH), 5.84 (d, J=11.4 Hz, 1H, ═CH), 6.30 (d, J=11.4 Hz, 1H, ═CH); ¹³C-NMR (200 MHz, CDCl₃) 12.28, 15.82, 20.87, 22.30, 23.43, 26.23, 26.92, 28.23, 28.87, 37.15, 40.32, 40.62, 42.15, 44.65, 45.68, 48.33, 56.06, 56.31, 67.16, 67.38, 72.40, 115.34, 123.75, 129.36, 131.25, 139.30, 142.78.

### Example 10

### Preparation of (1R,3R,7E,22E,24E)-24a-homo-19-nor-9,10-secoergosta-5,7,22,24-tetraen-1,3,25-triol (19)

In a round-bottom flask (10 mL) diisopropylamine (0.15 mL) in THF (2 mL) was placed. The mixture was cooled down to −60° C. and the solution of n-butyl lithium (1.6 M) (0.6 mL) was added dropwise. After 15 min. (7E)-(1R,3R)-24-phenylsulfonyl-1,3-bis(tert-butyldimethylsilyloxy)-9,10-seco-19,22,23-trinorchola-5,7-dien (7) (0.25 g) in THF (2 mL) was added. After next 15 min. ethyl 3-methyl-4-oxobut-2-enoate (17) (0.2 g) was added and the resulting mixture was stirred for 60 min. at −60° C. The reaction was quenched with brine (1 mL) addition, the reaction product was extracted with tetrahydrofurane (2×10 mL). Organic layer was dried over anh. MgSO₄. Solvents were removed under reduced pressure, the residue was dissolved in anh. ethyl ether and it was treated with methyl magnesium bromide (3M, 2 mL). After 30 min. brine (2 mL) was added and the reaction product was extracted with ethyl ether (3×10 mL). Organic phase was dried over anh. MgSO₄. The solvents were removed under vacuum, the residue was dissolved in THF (2 mL) and it was diluted with methanolic saturated solution of Na₂HPO₄ (3 mL). While vigorous stirring sodium amalgam (about 1 g) was added protionwise, until disappearance of the substrate (TLC). The mixture was filtered using pleated filter paper, the filtrate was condensed under reduced pressure, and to the residue water (5 mL) was added. The reaction product was extracted with t-butyl methyl ether (3×10 mL). Organic phase was dried over anh. MgSO₄. Solvents were removed, the residue was dissolved in the mixture of methanol-chloroform 1:1 (16 mL) and camphorsulfonic acid (90 mg) was added. The solution was stirred for 90 min. at RT. Sodium hydrogen carbonate (90 mg) was added into the reaction mixture and stirring was continued for 15 min. The solution was filtered through pleated filter paper, it was mixed with silica gel (1 g) and condensed on vacuum rotavapour. The residue was purified by silica gel chromatography (hexane—ethyl acetate gradient from 10% to 80%). The product 19 was obtained in about 60 mg yield.

UV λₘₐₓ 244.0, 251.8, 261.6 nm; ¹H-NMR (200 MHz, CDCl₃); δ 0.57 (s, 3H, CH₃), 0.92 (s, 6H, 2 x CH₃), 1.06 (d, J=6.8 Hz, 3H, CH₃), 1.96 (s, 3H, CH₃), 4.08 (m, 1H, CH—OH), 4.15 (m, 1H, CH—OH), 5.35 (bs, 1H, ═CH), 5.52 (m, 1H, ═CH), 5.82 (bs, 1H, ═CH), 5.96 (d, J=11.4 Hz, 1H, ═CH), 6.24 (d, J=11.4 Hz, 1H, ═CH).

### Example 11

### Preparation of (1R,3R,7E,22E,24E)-24a,26,27-trihomo-19-nor-9,10-secoergosta-5,7,22,24-tetraeno-1,3,25-triol (20)

In a round-bottom flask (10 mL) diisopropylamine (0.15 mL) in THF (2 mL) was placed. The mixture was cooled down to −60° C. and the solution of n-butyl lithium (1.6 M) (0.6 mL) was added dropwise. After 15 min. (7E)-(1R,3R)-24-phenylsulfonyl-1,3-bis(tert-butyldimethylsilyloxy)-9,10-seco-19,22,23-trinorchola-5,7-dien (7) (0.25 g) in THF (2 mL) was added. After next 15 min. ethyl 3-methyl-4-oxobut-2-enoate (17) (0.2 g) was added and the resulting mixture was stirred for 60 min. at −60° C. The reaction was quenched with brine (1 mL) addition and the reaction product was extracted with tetrahydrofurane (2×10 mL). Organic phase was dried over anh. MgSO₄. Solvents were removed under reduced pressure, the residue was dissolved in anh. ethyl ether (2 mL) and the resulting solution was added dropwise into ethyl magnesium bromide (3 M, 2 mL). After 30 min. brine (2 mL) was added and the reaction product was extracted with ethyl ether (3×10 mL). The organic phase was dried over anh. MgSO₄. Solvents were removed under vacuum and the residue was dissolved in THF (2 mL). The mixture was diluted with saturated solution of Na₂HPO₄ (3 mL) While vigorously stirring, sodium amalgam (ok. 1 g) was added portionwise, until the substrate entirely disappeared (TLC). The mixture was filtered using pleated filter paper, the filtrate was condensed under vacuum and to the residue water was added (5 mL). The product was extracted with t-butyl methyl ether (3×10 mL). Organic phase was dried over anh. MgSO₄. Solvents were removed under reduced pressure, the residue was dissolved in the mixture of methanol-chloroform 1:1 (16 mL), camphorsulfonic acid (90 mg) was added and the solution was stirred for 90 min. at RT. After sodium hydrogen carbonate (90 mg) addition stirring was continued for 15 min. The mixture was filtered using pleated filter paper, silica gel was added (1 g) and the solvents were removed under reduced pressure. The residue was purified by silica gel chromatography (hexane—ethyl acetate gradient from 10% to 80%). The product 19 was obtained in about 45 mg yield.

UV λₘₐₓ 244.2, 252.0, 261.6 nm; ¹H-NMR (200 MHz, CDCl₃); δ 0.58 (s, 3H, CH₃), 0.96 (s, 6H, 2 x CH₃), 1.08 (d, J=6.8 Hz, 3H, CH₃), 1.96 (s, 3H, CH₃), 4.12 (m, 1H, CH—OH), 4.18 (m, 1H, CH—OH), 5.34 (bs, 1H, ═CH), 5.50 (m, 1H, ═CH), 5.82 (bs, 1H, ═CH), 5.92 (d, J=11.4 Hz, 1H, ═CH), 6.23 (d, J=11.4 Hz, 1H, ═CH).

1. Synthone for the preparation of 19-nor vitamin D derivatives, represented by Formula (I), 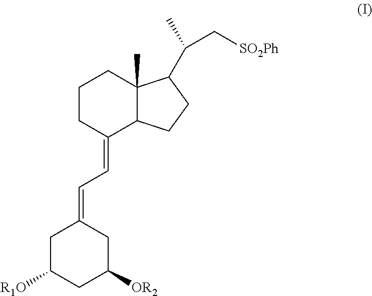
wherein R₁ and R₂ are the same or different and represent independently hydrogen atom or hydroxyl protecting group.

2. Synthone according to claim 1, wherein R₁ and R₂ represent the silyl group —Si(R₈)(R₉)(R₁₀), wherein R₈-R₁₀ are the same or different and represent C₁₋₆-alkyl or phenyl.

3. Synthone according to claim 1, which is (7E)-(1R,3R)-24-phenylsulfonyl-9,10-seco-19,22,23-trinorchola-5,7-dien-1,3-diol.

4. Synthone according to claim 3, which is in the crystalline form showing characteristic peaks in X-ray powder diffraction pattern recorded with CuKα, λ=1,54056A, as presented by the following reflection angles 2θ [°], interplanar spacings d [Å] and relative intensities in attitude to the most intensive diffraction peak, I/I₀ [%] as set forth in Table 1:
TABLE 1

| d [Å] | 2θ [°] | I/I₀ [%] |
|---|---|---|
| 10.208 | 8.66 | 1.1 |
| 9.734 | 9.08 | 14.5 |
| 7.729 | 11.44 | 3.4 |
| 7.515 | 11.77 | 28.6 |
| 6.128 | 14.44 | 9.3 |
| 5.843 | 15.15 | 25.1 |
| 5.525 | 16.03 | 100 |
| 5.437 | 16.29 | 41.4 |
| 5.150 | 17.21 | 5.4 |
| 4.681 | 18.94 | 22.8 |
| 4.522 | 19.62 | 25.6 |
| 3.856 | 23.05 | 2.7 |
| 3.777 | 23.54 | 24 |
| 3.460 | 25.73 | 29 |
| 3.431 | 25.95 | 2.8 |
| 3.333 | 26.72 | 5.5 |
| 3.296 | 27.03 | 15.1 |
| 3.176 | 28.07 | 5.2 |
| 3.025 | 29.51 | 0.9 |
| 2.294 | 39.24 | 1.1 |

5. Synthone according to claim 4, further characterized by X-ray powder diffraction pattern substantially as depicted in FIG. 1.

6. Synthone according to claim 4, further characterized by infrared spectrum (KBr) with characteristic peaks at λ: 3375, 3048, 2927, 2876, 2830, 1618, 1446, 1304, 1148, 1085, 1046, 1034, 978, 810, 749, 724, 689, 545 cm⁻¹.

7. Synthone according to claim 1, which is (7E)-(1R,3R)-24-phenylsulfonyl-1,3-bis(tert-butyldimethylsilyloxy)-9,10-seco-19,22,23-trinorchola-5,7-dien.

8. Process for preparation of synthone of Formula (I), 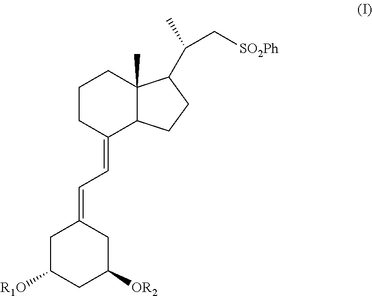
wherein R₁ and R₂ are the same or different and represent independently hydrogen atom or hydroxyl protecting group, characterized by that phosphine oxide derivative of Formula (II), 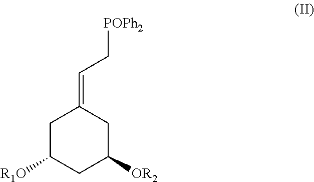
wherein substituents R₁ and R₂ represent independently hydrogen atom or hydroxyl protecting group, is reacted under Horner-Wittig-type reaction conditions with (7aR)-7a-methyl-1-((S)-1-(phenylsulfonyl)propan-2-yl)hexahydro-1H-inden-4(2H)-on (III).

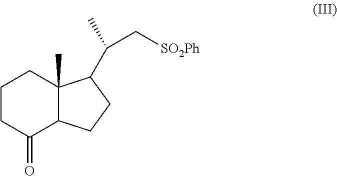

9. Process according to claim 8, characterized by that the phosphine oxide is (7E)-(1R,3R)-24-phenylsulfonyl-1,3-bis(tert-butyldimethylsilyloxy)-9,10-seco-19,22,23-trinorchola-5,7-dien.

10. The starting compound for the preparation of synthones of Formula (I), which is (7aR)-7a-methyl-1-((S)-1-(phenylsulfonyl)propan-2-yl)hexahydro-1H-inden-4(2H)-on.

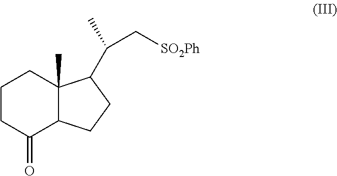

11. The compound according to claim 10, which is in the crystalline form showing characteristic peaks in X-ray powder diffraction pattern recorded with CuKα, λ=1,54056 Å, as presented by the following reflection angles 2θ [°], interplanar spacings d [Å] and relative intensities in attitude to the most intensive diffraction peak, I/I₀ [%] as set forth in Table 2:
TABLE 2

| d [Å] | 2θ [°] | I/I₀ [%] |
|---|---|---|
| 11.144 | 7.93 | 2.5 |
| 8.486 | 10.42 | 22.2 |
| 7.513 | 11.77 | 4.7 |
| 5.787 | 15.30 | 19.8 |
| 5.633 | 15.72 | 21.2 |
| 5.163 | 17.16 | 100 |
| 4.912 | 18.04 | 30.2 |
| 4.500 | 19.71 | 30.3 |
| 4.270 | 20.79 | 10.3 |
| 4.048 | 21.94 | 19.3 |
| 3.929 | 22.61 | 12.3 |
| 3.709 | 23.97 | 15.3 |
| 3.568 | 24.93 | 11.5 |
| 3.442 | 25.86 | 8.4 |
| 3.231 | 27.59 | 10.1 |
| 3.037 | 29.39 | 5.6 |
| 2.749 | 32.55 | 3.1 |
| 2.539 | 35.32 | 3.8 |
| 2.447 | 36.70 | 2.3 |

12. The compound according to claim 11, further characterized by X-ray powder diffraction pattern substantially as depicted in FIG. 2.

13. The compound according to claim 11 further characterized by infrared spectrum (KBr) with characteristic peaks at λ: 84, 3069, 2965, 2,51, 2903, 2881, 1698, 1448, 1303, 1241, 1145, 1088, 777, 749, 691, 589, 538, 510 cm⁻¹.

14. Use of synthone of Formula (I) according to claim 1 for preparation of biologically active 19-nor vitamin D derivatives of general Formula (IV), 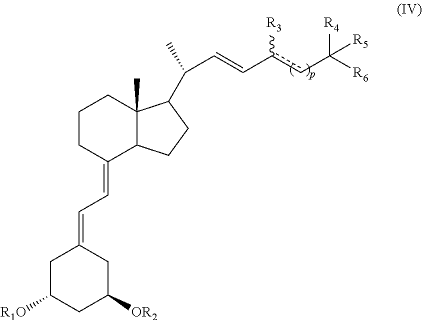
wherein [symbol] represents single or double bond, p represents an integer 0 to 3, R₁ and R₂ represent independently hydrogen atom or hydroxyl protecting group, R₃ represents hydrogen atom, CH₃ or hydroxyl group, R₄, R₅ and R₆ represent independently hydrogen atom, C₁-C₃-alkyl or hydroxyl group or two of R₄, R₅ and R₆ substituents altogether form cyclopropyl group.

15. Use according to claim 14 wherein 19-nor vitamin D derivatives of general Formula (IV), wherein p represents 0, [symbol] represents single bond, R₁ and R₂ represent hydrogen atoms, R₃, R₄ and R₅ represent CH₃ groups, R₆ represents hydroxyl group, and carbon atom C-24 has R or S configuration, are obtained.

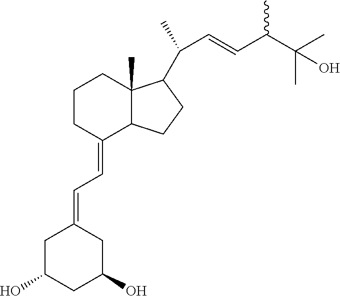

16. Use according to claim 15, wherein 19-nor vitamin D derivatives of general Formula (IV), wherein p represents 0, [symbol] represents single bond, R₁ and R₂ represent hydrogen atoms, R₃, R₄ and R₅ represent CH₃ groups, R₆ represents hydroxyl group, and carbon atom C-24 has S configuration, are obtained.

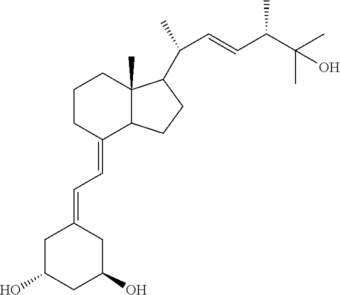

17. Process for preparation of biologically active 19-nor vitamin D derivatives of general Formula (IV), 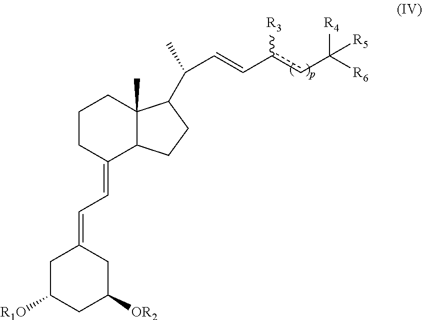
wherein [symbol] represents single or double bond, p represents an integer 0 to 3, R₁ and R₂ represent independently hydrogen atom or hydroxyl protecting group, R₃ represents hydrogen atom, CH₃ or hydroxyl group, R₄, R₅ and R₆ represent independently hydrogen atom, C₁-C₃-alkyl or hydroxyl group or two of R₄, R₅ and R₆ substituents altogether form cyclopropyl group, characterized in that it comprises:
(i) reacting synthone of Formula (I), 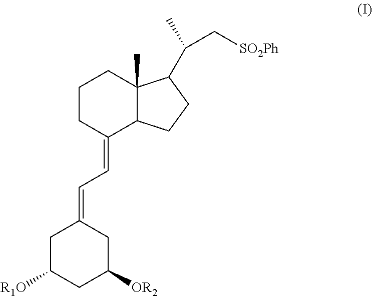
wherein R₁ and R₂ are the same or different and represent hydroxyl protecting groups,
with aldehyde of Formula (Va) or (Vb), 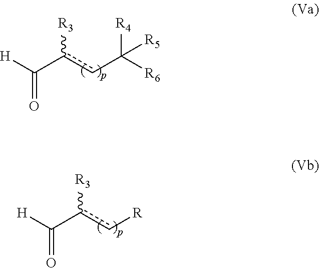
wherein p represents an integer 0 to 3, [symbol] represents single or double bond, R₃ represents hydrogen atom, CH₃ or hydroxyl group, R₄ and R₅ represent hydrogen atom or C₁-C₃-alkyl group, R₆ represents hydroxyl group and R represents carboxyl group or two of R₄, R₅ and R₆ substituents altogether form cyclopropyl group, in the presence of strong organic base in aprotic solvent, to obtain the mixture of alfa-hydroxysulfones,
(ii) reductive elimination of the adjacent phenylsulfonyl and hydroxyl groups from the mixture of alfa-hydroxysulfones obtained in step (ii) with sodium amalgam, to obtain the product of olefination of Formula (IVa) or (IVb), respectively, 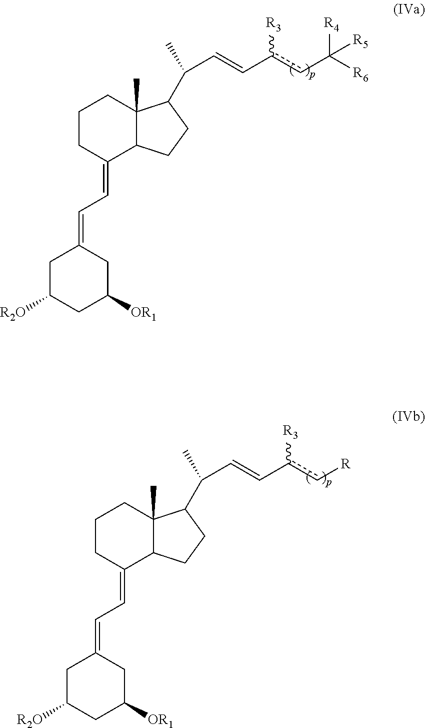
wherein R₁-R₆, R and p have the meaning as defined above,
(iii) optionally, standard synthetic modification of the side chain of the compound of Formula (IVb), to obtain the compound of Formula (IVa), wherein R₄-R₆ and p have the meaning as defined above,
(iv) optionally, removing the hydroxyl protecting groups and purification of the product.
